(19) United States
(12) Patent Application Publication
(10) Pub. No.: US 2024/0069497 A1
(43) Pub. Date: Feb. 29, 2024

(54) ELECTRONIC DEVICE, FORM DETERMINATION METHOD, AND RECORDING MEDIUM

(71) Applicant: CASIO COMPUTER CO., LTD., Tokyo (JP)

(72) Inventor: Hiroyasu IDE, Tokyo (JP)

(73) Assignee: CASIO COMPUTER CO., LTD., Tokyo (JP)

(21) Appl. No.: 18/456,213

(22) Filed: Aug. 25, 2023

(30) Foreign Application Priority Data
Aug. 26, 2022 (JP) 2022-134890

(51) Int. Cl. G04G 21/02; G01P 7/00; G01P 15/18; G04G 99/00

(52) U.S. Cl. CPC G04G 21/02; G01P 7/00; G01P 15/18; G04G 99/006

(57) ABSTRACT

An electronic device is an electronic device to be worn on an arm of a user, and includes a detector that detects exercise information of the user, and a controller. The controller determines, based on the exercise information detected by the detector, the form of the exercise of the user.

### CROSS-REFERENCE TO RELATED APPLICATIONS

This application claims the benefit of Japanese Patent Application No. 2022-134890, filed on Aug. 26, 2022, the entire disclosure of which is incorporated by reference herein.

### FIELD OF THE INVENTION

The present disclosure relates generally to an electronic device, a form determination method, and a recording medium.

### BACKGROUND OF THE INVENTION

In recent years, electronic devices that are worn on the arm and that detect the movement of the body of a user have been developed. Examples of such electronic devices include wearable terminals such as smartwatches, activity trackers, and the like. One technology used in such electronic devices involves calculating forward-backward acceleration of the user from acceleration data obtained by an acceleration sensor, and counting a number of steps by counting, as one step, points in time at which the calculated forward-backward acceleration is zero.

### SUMMARY OF THE INVENTION

One aspect of an electronic device according to the present disclosure is an electronic device to be worn on an arm of a user, the electronic device including:
a detector that detects exercise information of the user; and
a controller, wherein
the controller
   determines, based on the exercise information detected by the detector, a form of an exercise of the user.

### BRIEF DESCRIPTION OF DRAWINGS

A more complete understanding of this application can be obtained when the following detailed description is considered in conjunction with the following drawings, in which:

FIG. 1 is a block diagram illustrating an example of the functional configuration of an electronic device according to an embodiment;

FIG. 2 is a drawing for explaining various axes of acceleration detected by a detector in a case in which the electronic device according to the embodiment is worn on a left wrist of a user;

FIG. 3 is a drawing illustrating an example of the acceleration detected by the electronic device according to the embodiment;

FIG. 4 is an example of a flowchart of arm swing determination processing according to the embodiment;

FIG. 5 is a drawing for explaining x2 axis acceleration calculated in the arm swing determination processing according to the embodiment;

FIG. 6 is a drawing for explaining a horizontal acceleration ha and the like calculated in the arm swing determination processing according to the embodiment;

FIG. 7 is a drawing illustrating an example of a norm n and a leveling horizontal signal Lha calculated in the arm swing determination processing according to the embodiment;

FIG. 8 is a drawing illustrating an example of horizontal velocity obtained in the arm swing determination processing according to the embodiment;

FIG. 9 is a drawing illustrating an example of the acceleration detected by the electronic device according to the embodiment in a case in which the user walks while bending the arm at substantially a right angle;

FIG. 10 is a drawing illustrating an example of the norm n and the leveling horizontal signal Lha calculated in the arm swing determination processing according to the embodiment in a case in which the user walks while bending the arm at substantially a right angle; and

FIG. 11 is a drawing illustrating an example of horizontal velocity obtained in the arm swing determination processing according to the embodiment in a case in which the user walks while bending the arm at substantially a right angle.

### DETAILED DESCRIPTION OF THE INVENTION

Hereinafter, an electronic device and the like according to various embodiments are described while referencing the drawings. Note that, in the drawings, identical or corresponding components are denoted with the same reference numerals.

### Embodiments

An electronic device according to this embodiment is an information processing device that can be worn on an arm of a user and that can determine a form of an exercise by an acceleration sensor. In one example, the electronic device according to this embodiment is a smartwatch or an electronic watch.

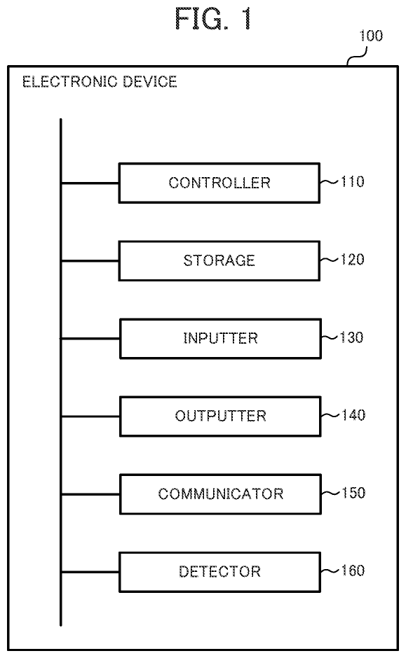

As illustrated in FIG. 1, an electronic device 100 includes a controller 110, a storage 120, an inputter 130, an outputter 140, a communicator 150, and a detector 160.

In one example, the controller 110 is configured from a processor such as a central processing unit (CPU) or the like. The controller 110 executes, by a program stored in the storage 120, processing for realizing the various functions of the electronic device 100, hereinafter described arm swing determination processing, and the like. The controller 110 is compatible with multithreading, and can execute a plurality of processes in parallel.

The storage 120 stores programs to be executed by the controller 110 and necessary data. The storage 120 can include random access memory (RAM), read-only memory (ROM), flash memory, and the like, but the storage 20 is not limited thereto. Note that the storage 120 may be provided inside the controller 110.

The inputter 130 is a user interface such as a push button switch, a touch panel, or the like, and receives operations/inputs from the user. When the inputter 130 includes a touch panel, the touch panel may be integrated with a display of the outputter 140.

The outputter 140 includes a display such as a liquid crystal display, an organic electro-luminescence (EL) display, or the like, and displays display screens, operation screens, and the like that provide the functions of the electronic device 100.

In one example, the communicator 150 is implemented as network interface that is compatible with a wireless local area network (LAN), long term evolution (LTE), or the like. The electronic device 100 can communicate with the internet and other devices via the communicator 150.

The detector 160 detects exercise information of the user. In the present embodiment, acceleration information is used as the exercise information of the user. Accordingly, the detector 160 includes an acceleration sensor that detects acceleration in each direction of three axes (X-axis, Y-axis, Z-axis) that are orthogonal to each other. It is preferable that, in the acceleration sensor, the acceleration is detected using a sampling frequency of about 25 Hz to 100 Hz. The controller 110 can acquire, as a detected value and at a desired timing (note that the temporal resolution depends on the sampling frequency), a value detected by the acceleration sensor of the detector 160.

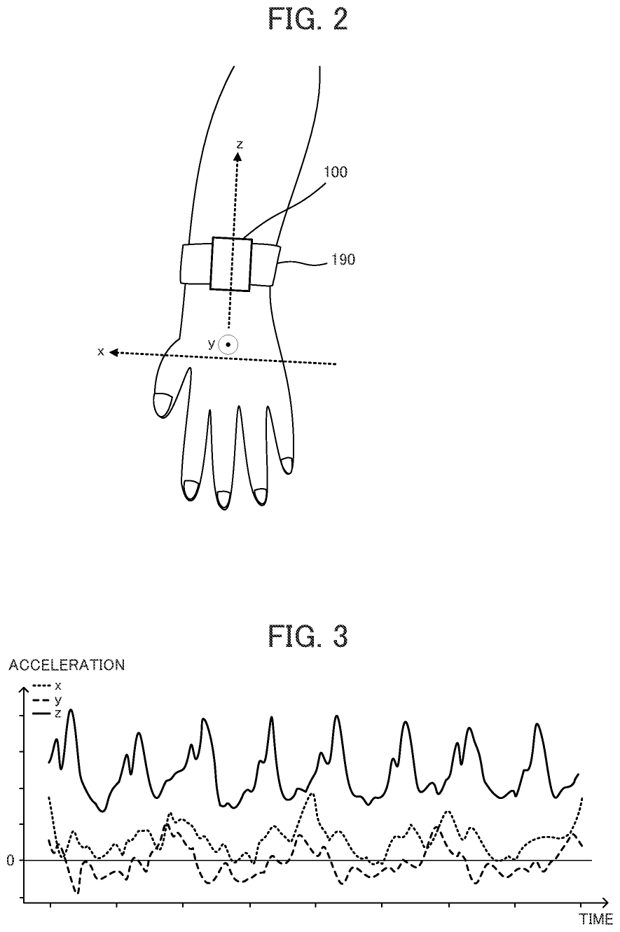

As illustrated in FIG. 2, the electronic device 100 is worn on a wrist of the user using a band 190. The arm on which the electronic device 100 is worn may be the right arm or the left arm but, in this case, as illustrated in FIG. 2, a case is described in which the electronic device 100 is worn on the back side of the left hand. Note that whether the electronic device 100 is worn on the left or right arm may be input via the inputter 130 or the like and stored in the storage 120.

In this case, the various axes of the acceleration sensor of the detector 160 are, as illustrated in FIG. 2, an x axis, which is a direction from the little finger toward the thumb, a y axis, which is a direction from the side of the wrist with the palmaris longus muscle (palm side) toward the back side thereof (the back side of the hand), and a z axis, which is a direction from the hand toward the elbow. Note that, although gravity is a downward force, gravitational acceleration is indistinguishable from acceleration that occurs when an object is accelerating upwards at 1G in zero gravity space. As such, the acceleration sensor detects the gravitational acceleration as vertically upward acceleration.

When, as illustrated in FIG. 2, the user walks while wearing the electronic device 100, the acceleration on each axis is acquired by the detector 160 as a discrete signal sampled at the sampling frequency. When the user walks without bending the elbow, the acceleration on each axis is acquired as illustrated in FIG. 3, for example. In this case, frequently, the z axis value that is affected by the gravitational acceleration is the greatest, and the x axis value that is affected by the forward-backward swinging of the hand is the next greatest. Additionally, since the hand rarely swings from left to right when walking, frequently, the y axis value is comparatively less.

Accordingly, in such a case, it is not strange to think that an arm swing direction can be determined on the basis of the x axis acceleration value. However, in reality, due to shifting of a wearing position of the electronic device 100, the manner in which the user swings their arm, and the like, the x axis may not reflect only the forward-backward swinging of the arm. As such, when determining the arm swing direction, the electronic device 100 splits the values of the acceleration on the three axes detected by the detector 160 into a vertical direction (gravitational direction) and a direction orthogonal to the vertical direction (horizontal direction), acquires a horizontal velocity from the latter value (horizontal acceleration), and determines the arm swing direction on the basis of this horizontal velocity.

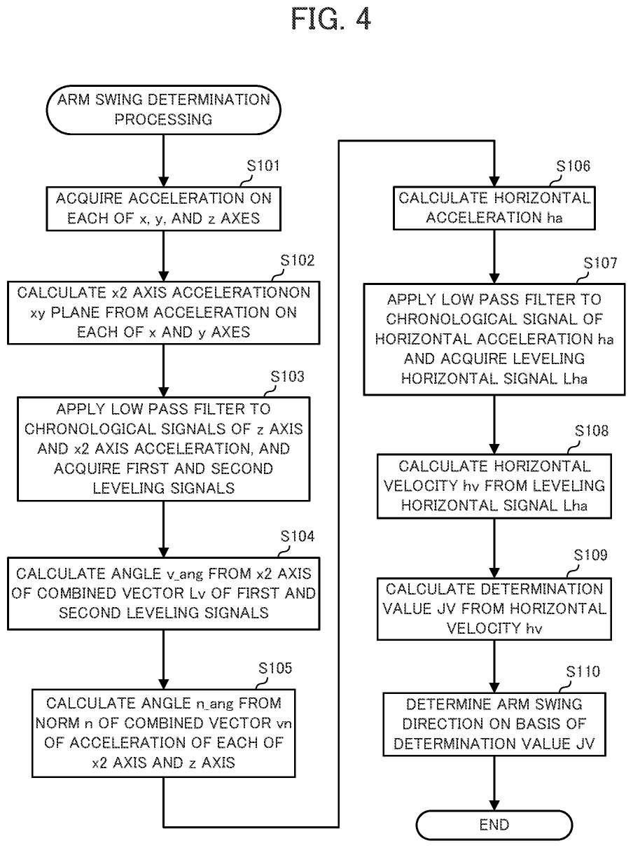

Arm swing determination processing for determining the arm swing direction on the basis of the values of the acceleration on the three axes is described while referencing FIG. 4. This processing may be executed in parallel with other necessary processings when the electronic device 100 is started up, or may be executed upon receipt of a command input by the user via the inputter 130 to perform the arm swing determination.

Firstly, the controller 110 acquires, from the detector 160, an acceleration value of each of the x axis, they axis, and the z axis (x axis signal, y axis signal, and z axis signal) (step S101). Next, the controller 110 calculates, from the x axis component (x axis signal) and the y axis component (y axis signal) of the acceleration, an x2 axis acceleration (x2 signal) on the xy plane (step S102).

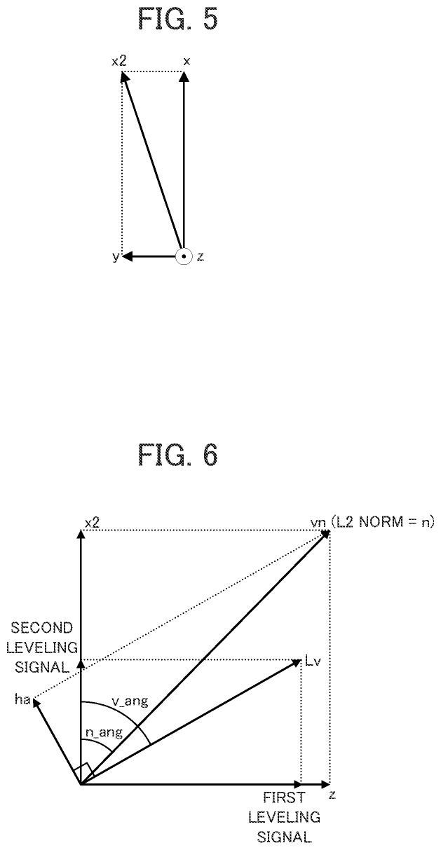

As illustrated in FIG. 5, the x2 axis acceleration is a sum vector of the x axis direction vector and the y axis direction vector of the acceleration, and this sum vector is orthogonal to the z axis. Moreover, a value (scalar value) of the x2 axis acceleration (x2 signal) is an L2 norm of a vector obtained by combining the x axis direction and the y axis direction of the acceleration. For the sake of convenience, the reference symbol of the x axis acceleration is applied as the reference symbol of the x2 axis acceleration (x2 signal). That is, when the x axis signal at a time i is expressed as xi and the y axis signal at the time i is expressed as yi, the x2 signal (x2i) is calculated by Equation (1) below. Note that sign(x) is a function that returns 1 when the reference symbol of x is positive and −1 when the reference symbol of x is negative.

x2i=√(xi²+yi²)×sign(xi) (1)

Next, the controller 110 applies a low pass filter (LPF) that cuts high-frequency components greater than or equal to a first reference frequency (0.05 Hz to 1 Hz, for example, 0.1 Hz) to each of a signal (first signal) expressing chronological data of the z axis acceleration (first acceleration component that is an acceleration component of a first axis (z axis) selected from the three axes), and a signal (second signal) expressing chronological data of the x2 axis acceleration (second acceleration component that is a component of the sum of the two axes (the x axis and the y axis) other than the first axis), and acquires a first leveling signal (LPF-applied zi) and a second leveling signal (LPF-applied x2i) (step S103).

Then, the controller 110 calculates an angle v_ang from the x2 axis of a leveling vector Lv that is a combined vector of a vector expressed by the first leveling signal and a vector expressed by the second leveling signal (step S104). That is, when the x2 signal at the time i is expressed as x2i and the z axis signal at the time i is expressed as zi, v_angi is calculated by Equation (2) below.

v_angi=tan⁻¹(LPF-applied zi/LPF-applied x2i) (2)

Next, the controller 110 calculates n, which is the L2 norm of the combined vector vn of the x axis signal (first acceleration component) and the x2 axis signal (second acceleration component), and n_ang, which is an angle from the x2 axis of vn (step S105). That is, when the x2 signal at the time i is expressed as x2i and the z axis signal at the time i is expressed as zi, ni and n_angi are calculated by Equations (2) and (3) below.

ni=√(x2i²+zi²) (3)

n_angi=tan⁻¹(zi/x2i) (4)

Next, the controller 110 calculates a horizontal acceleration ha by taking the component of the direction in which the combined vector vn of the z axis signal (first acceleration component) and the x2 axis signal (second acceleration component) is orthogonal to the v_ang on the x2-z plane (step S106). That is, when the norm n at the time i is expressed as ni, the v_ang at the time i is expressed as v_angi, and the n_ang at the time i is expressed as n_angi, hai is calculated by Equation (5) below.

hai=ni×sin(n_angi−v_angi)×(−1) (5)

The relationships between the various values described above are illustrated in FIG. 6. By applying the low pass filter to the x axis acceleration (first acceleration component) and the x2 axis acceleration (second acceleration component), various types of noise components caused by walking and arm swinging are removed and the gravitational acceleration becomes the dominant value. As such, the leveling vector Lv, which is the combined vector of the vector expressed by the first leveling signal and the vector expressed by the second leveling signal, becomes a vertical (average gravitational direction) vector. Accordingly, a vector ha, which is a vector perpendicular to the leveling vector Lv can be thought of as a horizontal vector, and it is understood that the horizontal acceleration is expressed by the vector ha.

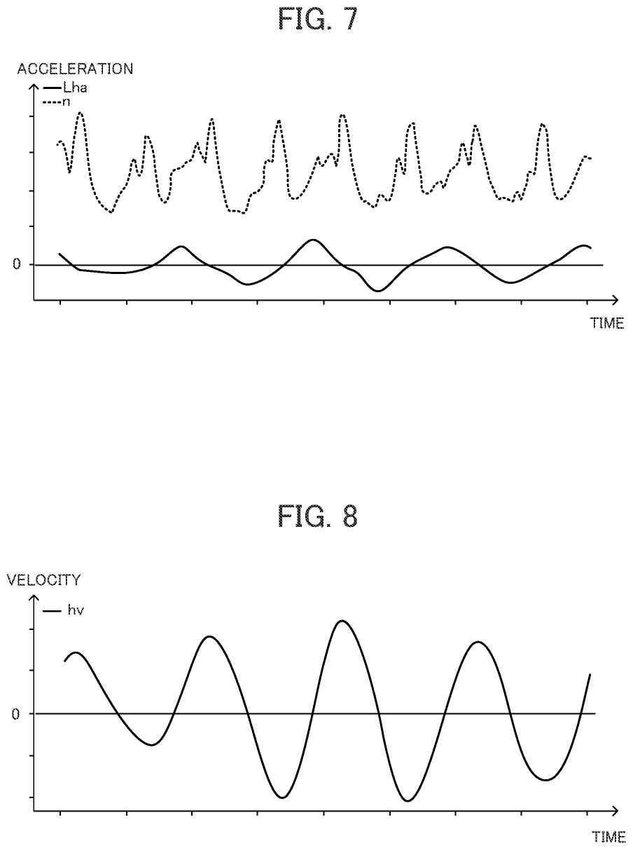

Next, in order to remove the high-frequency noise components of the horizontal acceleration ha, the controller 110 applies a low pass filter that cuts high-frequency components greater than or equal to a second reference frequency (1.5 Hz to 3 Hz, for example, 2 Hz) to a signal expressing chronological data of the horizontal acceleration ha, and acquires a leveling horizontal signal Lha (step S107). For example, when the norm n of the combined vector vn and the leveling horizontal signal Lha are calculated from the signal (chronological signal) expressing the chronological data of the acceleration on the three axes illustrated in FIG. 3, a graph such as illustrated in FIG. 7 is obtained.

Next, the controller 110 integrates the leveling horizontal signal Lha to calculate a horizontal velocity hv (step S108). Note that the leveling horizontal signal Lha also includes, as noise components, signals other than horizontal signals, and signals caused by walking or running (caused by movement of the user). As such, in order to suppress the influence of these noise components, the controller 110 may calculate a horizontal acceleration moving average (MAha) that is a moving average of the leveling horizontal signal Lha, and may calculate the horizontal velocity hv by subtracting the horizontal acceleration moving average MAha from the leveling horizontal signal Lha and then integrating.

Then, the controller 110 calculates a determination value JV for determining the arm swing direction from the horizontal velocity hv (step S109). Here, the controller 110 may use the horizontal velocity hv as the determination value JV without modification. However, the horizontal velocity hv also includes, as noise components, components other than the horizontal components, and components caused by walking or running (caused by movement of the user). Accordingly, in order to suppress the influence of these noise components, the controller 110 may calculate a horizontal velocity moving average MAhv that is a moving average of the horizontal velocity hv, and may calculate the determination value JV by subtracting the horizontal velocity moving average MAhv from the horizontal velocity hv.

Next, the controller 110 determines the arm swing direction on the basis of the determination value JV (step S110), and ends the arm swing determination processing. A value of the determination value JV (that is, the horizontal velocity hv) takes a positive value when the arm is moving in the forward direction, and takes a negative value when the arm is moving in the backward direction. Moreover, the value is 0 at the point in time of a turn-back of the arm swing. For example, when calculating the horizontal velocity hv from the leveling horizontal signal Lha illustrated in FIG. 7, a graph such as illustrated in FIG. 8 is obtained. In this graph, the arm swing direction switches at the timing at which the value is 0, the arm swing direction is the forward direction while the value is positive, and the arm swing direction is the backward direction while the arm swing direction is negative. Accordingly, the controller 110 can determine the arm swing direction on the basis of the value of the horizontal velocity hv (that is, the determination value JV).

Furthermore, the controller 110 can determine a ground contact foot from a ground contact timing of the foot and the determination value JV. For example, the controller 110 determines that a foot on the same side as the arm on which the electronic device 100 is worn is contacting the ground when, in a period from a ground contact timing of one foot to a ground contact timing of the other foot, an amount of time that the determination value JV takes a positive value is longer than an amount of time that the determination value JV takes a negative value.

Additionally, provided that whether the arm on which the electronic device 100 is worn is the left or the right is stored in advance in the storage 120 by inputting via the inputter 130 or the like, the ground contact foot can be determined (estimated) on the basis of at least the value that the determination value JV takes. For example, when the arm on which the electronic device 100 is worn is the left arm and the user is walking, a determination (estimation) is made that the left foot is the ground contact foot when the determination value JV switches from 0 to positive. Additionally, there is a high possibility that both feet are contacting the ground at the moment at which the determination value JV switches from 0 to positive, but the right foot ceases to be a ground contact foot when the determination value JV increases and can be determined (estimated) to be a ground contact foot again when the determination value JV decreases and the determination value JV becomes 0 again. Moreover, a determination (estimation) may be made that the arm is at the side of the body of the user when the determination value JV takes an extreme value. Regarding the aforementioned, a similar determination (estimation) can be performed when the determination value JV takes a negative value or when the wearing arm is the right arm.

Note that any method can be used to detect the ground contact of the foot. For example, the controller 110 can detect the ground contact of the foot by detecting a peak in a combined signal of the accelerations of all three axes detected by the detector 160. In such a case, the controller 110 also functions as a ground contact detector.

Due to the arm swing determination processing described above, the controller 110 can determine the arm swing direction of the user on the basis of the data of the acceleration on the 3 axes. Additionally, the controller 110 can determine the ground contact foot by detecting the ground contact timing of the foot.

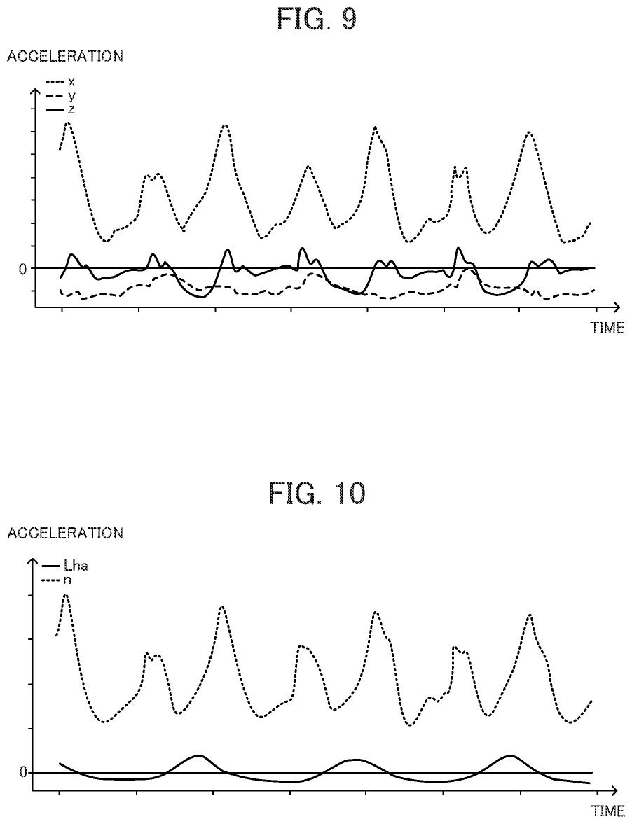

In the description given above, a case is described in which the user walks without bending the elbow. Next, a case is considered in which the user bends the elbow (for example, bends the arm at substantially a right angle) and walks. In this case, the x axis is most affected by the gravitational acceleration, the z axis is affected by the forward-backward swing of the arm, and acceleration on the various axes is acquired as illustrated in FIG. 9, for example.

Moreover, as understood from FIGS. 5 and 6, the magnitude of the norm n is greatly affected by the axis with the greatest amplitude among the three axes, the x axis, the y axis, and the z axis. Accordingly, a graph of the norm n along a time series is, as illustrated in FIG. 10, a graph that is similar to the x axis acceleration of FIG. 9.

Additionally, the horizontal acceleration ha is a vector perpendicular to the leveling vector Lv but, in this case as well, the various types of noise components are removed from the leveling vector Lv, and the gravitational acceleration becomes the dominant value. As such, the leveling vector Lv becomes a vertical (average gravitational direction) vector. Accordingly, even when the user bends the elbow and walks, a value of a vector perpendicular to the vertical direction (Lv) is obtained as the horizontal acceleration ha, and the controller 110 can acquire the leveling horizontal signal Lha such as illustrated in FIG. 10, for example, by leveling that value.

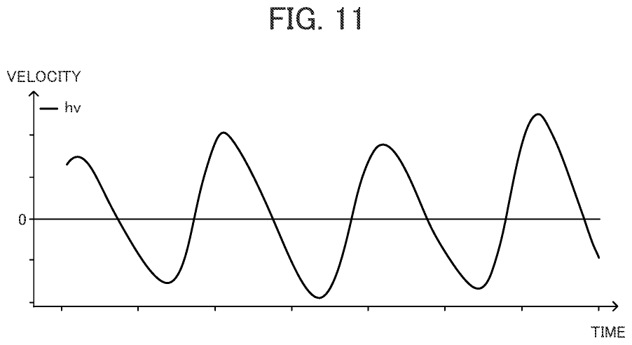

Moreover, the controller 110 can calculate the horizontal velocity hv such as illustrated in FIG. 11, for example, by integrating the leveling horizontal signal Lha.

Thus, even when the first axis is not the axis most affected by gravity (the z axis when the elbow is not bent, and the x axis with the elbow is bent), a substantially vertical vector can be obtained by calculating the leveling vector Lv, and the horizontal acceleration ha can be obtained by calculating a vector perpendicular to the leveling vector Lv.

Note that the technology described in Unexamined Japanese Patent Application Publication No. 2011-90548, which is a prior art document, calculates the forward-backward acceleration of the user from acceleration data obtained by an acceleration sensor, and counts the number of steps by counting, as one step, points in time at which the calculated forward-backward acceleration is zero. In reality, accurately calculating the forward-backward acceleration is difficult, but it is sufficient to detect the inversion of the reference numeral of the forward-backward acceleration to count the number of steps and, as such, it is possible to count the number of steps by this method without any problems. However, although the number of steps can be counted, this sort of method is unsuitable for determining (estimating) the actual form of the exercise. The present disclosure is made with the view of this situation, and enables the determination of the form of the exercise of the user on the basis of exercise information of the user.

### MODIFIED EXAMPLES

Note that, in the embodiment described above, acceleration is used as the exercise information of the user, but the exercise information of the user is not limited to acceleration. For example, a configuration is possible in which angular velocity is used as the exercise information of the user. When using angular velocity as the exercise information, the detector 160 is provided with a gyrosensor. Additionally, the controller 110 determines the arm swing direction of the user on the basis of the angular velocity of the arm detected by the gyrosensor. A configuration is possible in which both acceleration and angular velocity are used as the exercise information of the user. In such a case, the controller 110 can improve the accuracy of the arm swing determination by comparing the arm swing direction determined on the basis of the acceleration and the arm swing direction determined on the basis of the angular velocity.

Additionally, in the embodiment described above, a determination of the arm swing direction and a determination of the ground contact foot were performed as the determination of the form of the exercise of the user. However, a configuration is possible in which the controller 110 performs a form analysis (displaying the form of the exercise as an animation, offering improvement advice, or the like) of the exercise of the user on the basis of information about the determined arm swing and/or ground contact foot. Moreover, a configuration is possible in which the controller 110 sends the determined arm swing and/or ground contact foot, form analysis results, and the like to another device (smartwatch, smartphone, or the like) via the communicator 150, and displays the determined arm swing and/or contact foot, form analysis results, and the like on a display of the other device.

The electronic device 100 is not limited to a smartwatch, and can be realized by a smartphone provided with the detector 160, or a computer such as a portable tablet, a personal computer (PC), or the like. Specifically, in the embodiment described above, an example is described in which the program of the arm swing determination processing and the like executed by the controller 110 are stored in advance in the storage 120. However, a computer may be configured that is capable of executing the various processings described above by storing and distributing the programs on a non-transitory computer-readable recording medium such as a compact disc read-only memory (CD-ROM), a digital versatile disc (DVD), a magneto-optical, disc (MO), a memory card, and a USB memory, and reading out and installing these programs on the computer.

Furthermore, the program can be superimposed on a carrier wave and applied via a communication medium such as the internet. For example, the program may be posted to and distributed via a bulletin board system (BBS) on a communication network. Moreover, a configuration is possible in which the various processings described the above are executed by starting the programs and, under the control of the operating system (OS), executing the programs in the same manner as other applications/programs.

Additionally, a configuration is possible in which the controller 110 is constituted by a desired processor unit such as a single processor, a multiprocessor, a multi-core processor, or the like, or by combining these desired processors with processing circuitry such as an application specific integrated circuit (ASIC), a field-programmable gate array (FPGA), or the like.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled

1. An electronic device to be worn on an arm of a user, the electronic device comprising:
a detector that detects exercise information of the user; and
a controller, wherein
the controller
determines, based on the exercise information detected by the detector, a form of an exercise of the user.

2. The electronic device according to claim 1, wherein
the detector detects, as the exercise information, acceleration components on three axes that are orthogonal to each other, and
the controller
acquires, based on the acceleration components on the three axes detected by the detector, a horizontal velocity that is a horizontal velocity component, and
with the acquired horizontal velocity as a determination value determines, based on the determination value, an arm swing direction of the user.

3. The electronic device according to claim 2, wherein
the controller
splits the acceleration components on the three axes into a first acceleration component that is an acceleration component on a first axis selected from the three axes, and a second acceleration component that is a component of a sum of the acceleration components of two axes other than the first axis, and
acquires, based on the first acceleration component and the second acceleration component, the horizontal velocity.

4. The electronic device according to claim 3, wherein
the controller
calculates, based on a first leveling signal obtained by cutting high-frequency components greater than or equal to a first reference frequency in a first signal that is a signal expressing chronological data of the first acceleration component and a second leveling signal obtained by cutting the high-frequency components greater than or equal to the first reference frequency in a second signal that is a signal expressing chronological data of the second acceleration component, a horizontal acceleration, and
acquires the horizontal velocity from the calculated horizontal acceleration.

5. The electronic device according to claim 4, wherein
the controller
acquires the horizontal velocity from a leveling horizontal signal obtained by cutting high-frequency components greater than or equal to a second reference frequency in a signal expressing chronological data of the horizontal acceleration.

6. The electronic device according to claim 5, wherein
the controller
calculates a horizontal acceleration moving average that is a moving average of the leveling horizontal signal, and
acquires the horizontal velocity by integrating a value obtained by subtracting the horizontal acceleration moving average from the leveling horizontal signal.

7. The electronic device according to claim 6, wherein
the controller
calculates a horizontal velocity moving average that is a moving average of the horizontal velocity, and
sets a value obtained by subtracting the horizontal velocity moving average from the horizontal velocity as the determination value.

8. The electronic device according to claim 2, wherein the controller determines, based on at least the determined arm swing direction, a ground contact foot of the user.

9. The electronic device according to claim 8, wherein
the controller detects a timing of a ground contact of a foot of the user, and
determines, based on the detected timing of the ground contact and the determined arm swing direction, the ground contact foot of the user.

10. The electronic device according to claim 9, wherein
the controller
determines that a foot on a same side as the wearing arm is making ground contact when, in a period from the detected timing of the ground contact to a timing of a next ground contact, an amount of time in which the determination value takes a positive value is greater than an amount of time in which the determination value takes a negative value.

11. A form determination method in which an information processing device is worn on an arm of a user and includes a detector that detects exercise information of the user, and a controller, the method comprising:
determining, by the controller and based on the exercise information detected by the detector, a form of an exercise of the user.

12. A non-transitory recording medium storing a program that causes a controller, of an information processing device that is worn on an arm of a user and that includes a detector that detects exercise information of the user and the controller, to execute processing for:
determining, based on the exercise information detected by the detector, a form of an exercise of the user.
